(19) United States
(12) Patent Application Publication
(10) Pub. No.: US 2008/0090223 A1
(43) Pub. Date: Apr. 17, 2008

(54) Method for Diagnosing Hepatic Diseases

(76) Inventors: Fernando Jose Corrales Izquierdo, Pamplona (Navarra) (ES); Joaquin Fernandez Irigoyen, Pamplona (Navarra) (ES); Enrique Santamaria Martinez, Pamplona (Navarra) (ES); Javier Munoz Peralta, Pamplona (Navarra) (ES); Laura Sesma Aguirre, Pamplona (Navarra) (ES); Jesur Prieto Valtuena, Pamplona (Navarra) (ES); Matias Antonio Avila Zaragoza, Pamplona (Navarra) (ES)

Correspondence Address: WENDEROTH, LIND & PONACK, L.L.P., 2033 K STREET N. W., SUITE 800, WASHINGTON, DC, 20006-1021, US

(21) Appl. No.: 11/793,230

(22) PCT Filed: Feb. 15, 2005
(86) PCT No.: PCT/ES05/00074

§ 371 Date: Oct. 10, 2007

(30) Foreign Application Priority Data
Dec. 16, 2004 (ES) P 200402989

(51) Int. Cl. C12Q 1/00; G01N 33/92

(52) U.S. Cl. 435/4; 436/71

(57) ABSTRACT

The invention concerns a method for early diagnosis of hepatic diseases, which consists in determining in the samples of the diseased subjects, the presence of a apolipoprotein AI (Apo-AI) isoform, having oxidised W50, W108, and M112 residues. The inventive method is useful for diagnosing hepatocellular carcinoma and hepatitis B.

### TECHNICAL FIELD OF THE INVENTION

This invention is included within the field of the detection of oxidised forms of apolipoprotein AI.

### STATE OF THE ART PRIOR TO THE INVENTION

Hepatocellular carcinoma (HCC) is the neoplastic disease with the fifth highest incidence and the third cause of cancer death, with over 500,000 new cases diagnosed every year. Although the main causes of HCC are known, from them, infection by hepatitis B virus (HBV) or C virus (HCV), the consumption of food contaminated with aflatoxin, or abusive alcohol consumption, the prognosis for HCC patients is bad due to the aggressiveness of the lesion at the time of diagnosis and the lack of effective therapies. The identification of biomarkers which make it possible to effectively describe the stage of this disease is, therefore, of great interest.

There is evidence that many pathological processes are associated with quantitative and functional changes in the molecular constituents of body fluids. Excluding cellular components, the comparative analysis of body fluid samples from healthy and ill donors is only possible at the proteomic level and not at the transcriptional level. Although cerebrospinal fluid and urine are used in diagnostic medicine, there is increasing interest in the human serum proteome due to the fact that the serum constantly penetrates the tissues and, therefore, the beginning or the presence of a disease could be determined by measuring and characterising the thousands of individual circulating proteins and peptides. Moreover, serum determination techniques are bloodless and non-invasive, have good sample availability and are easy to perform, quick and inexpensive.

Traditional techniques which make it possible to diagnose HCC, such as the increase in alpha-fetoprotein (AFP) levels, are not effective in all cases and are positive when the stage of the disease is too advanced. Studies have been conducted to identify HCC markers using high-performance proteomic techniques, both in the liver (Zeindl-Eberhart E. et al. Hepatology (2004) 39:540-549), which would entail using invasive techniques to perform the diagnosis, and in serum (Steel F. L. et al. Proteomics (2003) 3:601-609; Quina Yu He et al. Proteomics (2003) 3:666-674). However, although this technology has proven effective in the detection of molecular targets related to the development of various diseases, its methodological complexity makes its use in clinical practise unthinkable. None of the above-mentioned studies provide information which may serve as the basis for the development of diagnostic methodologies applicable to clinical practise. On the other hand, various studies, including the above-mentioned ones, have established a relationship between the decrease in Apo AI levels and the development of liver diseases. However, the isoform of Apo AI, and its increase in the serum of patients suffering from hepatocellular carcinoma, which is the subject of this invention, has not been previously described.

### OBJECT OF THE INVENTION

The subject of this invention is the detection in a biological sample, preferably serum, of an increase in an acidic apolipoprotein AI isoform, oxidised in at least a tryptophan or methionine residue. Specifically, an apolipoprotein AI isoform oxidised in residues W50 and W108.

### DETAILED DESCRIPTION OF THE INVENTION

The present invention relates to a procedure to detect the increase in oxidised apolipoprotein AI isoforms in at least one tryptophan or methionine residue, or fragments thereof which contain said oxidised residues. In a preferred embodiment of the invention, it comprises the detection of the increase in at least one oxidised apolipoprotein AI isoform in at least one residue selected from W50, W108, and M112, or fragments thereof which contain said residues. Residues W50 and W108 may be oxidised to kynurenine, formylkynurenine, hydroxytryptophan, or 3-OH-kynurenine. On the other hand, residue M112 may be oxidised to M112 sulfoxide, or methionine sulfone.

Moreover, the procedure object of this invention is characterised in that said detection is performed on isolated biological samples. With these samples being selected from: serum, plasma, blood, urine, saliva, cerebrospinal fluid, tears, amniotic fluid, tissue wash, tissue homogenate and cell lysate. In a preferred embodiment of the invention, said biological sample is serum.

In a specific embodiment of this invention, the detection of oxidised apolipoprotein AI isoforms is performed on samples from individuals with hepatitis, and preferably individuals with hepatitis B (HBV).

As used in this invention, the term “individual” relates to any animal, preferably a mammal. In a preferred embodiment of the invention, the individual is a man or a woman.

In the procedure disclosed in this invention, the detection of said increase may be conducted by comparing with purified Apo AI standards in known quantities, or by comparing with the quantities of said isoform present in the same type of biological sample obtained from healthy control individuals.

In a preferred embodiment, the increase of the oxidised form in the serum with respect to the control level observed by means of said procedure is at least 1.5-fold, and preferably at least 2.5-fold.

In addition, the present invention is characterised in that it comprises the use of methods to detect and quantify the presence of oxidised Apo AI isoforms selected from: mass spectrometry, immunoassays, chemical assays, liquid chromatography, direct and indirect photometric methods, and combinations thereof. In a preferred embodiment, the mass spectrometry methods are selected from: tandem mass spectrometry coupled with liquid chromatography (LC-MS) and MALDI-TOF-MS (matrix-assisted laser desorption/ionization mass spectrometry time-of-flight MS). Moreover, in a preferred embodiment of the invention, the immunoassays are selected from: homogeneous assays, heterogeneous assays, enzyme immunoassays (EIA, ELISA), competition assays, immunometric assays (sandwich), turbidimetric assays, nephelometric assays and combinations thereof; which are extensively described in “The Immunoassay Book”, edited by David Wild, 2^{nd} Edition 2001, Nature Publishing Group, which is included as a reference. In a preferred embodiment, the methods of detection and quantification of oxidised forms of apolipoprotein AI comprise the use of antibodies, aptamers and/or lecithins which specifically recognise said Apo AI isoforms and fragments thereof.

This invention also makes it possible to relate said increase in apolipoprotein AI isoforms to the presence of hepatocellular carcinoma in the individual from whom the biological sample has been obtained. In a specific embodiment, said hepatocellular carcinoma is at an early stage.

Moreover, this invention relates to a kit for the determination of the increase in apolipoprotein AI isoforms described above, characterised in that it comprises reagents to conduct the methods used in this invention. In a preferred embodiment, said reagents are selected from specific ligands of Apo AI isoforms, marker components to detect Apo AI isoforms, buffers, diluents, standards and controls. In a preferred embodiment, the kit additionally comprises bottles, phials, tubes, needles, solid substrates and instructions.

In another respect, the present invention relates to a procedure for in vitro diagnosis of hepatocellular carcinoma, characterised in that it comprises the detection of the increase in oxidised apolipoprotein AI isoforms in at least one tryptophan or methionine residue, or fragments thereof which contain at least one of said oxidised residues. Said diagnostic procedure is characterised in that it detects said oxidised apolipoprotein AI isoforms as previously described in this section.

### BRIEF DESCRIPTION OF THE FIGURES

FIG. 1: Identification of the increase in Apo AI and Apo AIV levels in serum of MATIA−/− mice. Serum samples from control and MATIA−/− mice, 1, 3, 5, 12, and 18 months of age, were analysed by means of two-dimensional electrophoresis. Panel A shows representative gels from three independent analyses. The differential bands are identified in areas 1 and 2. Panel B shows the typical spectrum obtained by means of MALDI TOF MS from tryptic digestions of bands 1 and 2. The peptides whose sequence coincides with that of Apo AIV (1) and Apo AI (2) sequences are indicated.

FIG. 2: Identification of specific residues of methionine sulfoxide on isoform 1 of Apo AI by means of ESI-MS/MS. Tryptic peptides of the Apo AI species were resolved by means of reverse-phase nanoflow liquid chromatography. The column was connected to a tandem mass spectrometer by means of an electrospray source. Fragmentation spectra of the peptides originating from isoform 1, which contained derivatives of methionine sulfoxide in positions 85 and 116, are shown. Modifications were confirmed in three independent experiments. In all the assays, isoforms 2 and 3 showed residues M85 and M116 to be non-oxidised.

FIG. 3: Analysis of Apo AI isoforms in human liver diseases by means of two-dimensional electrophoresis. AI isoforms in serum of patients suffering from the above-mentioned liver pathologies were analysed. In all groups, serum samples from 6 patients were analysed, with the sole exception of NASH (non-alcoholic steatohepatitis), in which 15 patients were included in the study. Panel A shows representative 2D gel sections which contain four Apo AI species. The protein bands were identified by means of MALDI TOF mass spectrometry and de novo peptide sequencing. Densitometry of the Apo AI bands for those groups which exhibited a different isoform distribution than the control individuals is shown in panel B. * and ** indicate p<0.05, as compared to control individuals, in accordance with Student's t-test.

FIG. 4: Identification of specific derivatives of methionine sulfoxide and formylkynurenine in isoform 1 of human Apo AI. Tryptic digestions of Apo AI isoforms from control patients and patients suffering from HBV+HCC were resolved by means of nano-LC chromatography. Eluted peptides were characterised by means of tandem mass spectrometry (ESI/MS/MS). The spectra of the peptides with specific methionine sulfoxide (M112) and formylkynurenine (W50 and W108) ions are shown. The modifications were confirmed in three independent experiments and were only detected in isoform 1. Isoforms 2, 3 and 4 showed non-oxidised methionine and tryptophan residues.

### EXAMPLE 1

In this study, MATIA−/− mice were used, characterised by a chronic reduction in hepatic AdoMet enzyme, which induces the spontaneous development of non-alcoholic steatohepatitis (NASH) and cellular hepatocarcinoma at 18 months of age.

Serum samples were taken from control and MATIA−/− mice, 3, 5, 12, and 18 months of age, and were subsequently analysed by means of two-dimensional electrophoresis (2DE), followed by mass spectrometry. In this way, an average resolution of 500 proteins was obtained, and a differential comparison was conducted by means of PDQUEST, in which increases or decreases of at least two-fold were accepted as differences. Using this criterion, only two proteic bands were obtained from the serum of MATIA−/− mice, whose increase was consistent in all assays. Furthermore, these differences were observed starting with mice 3 months of age.

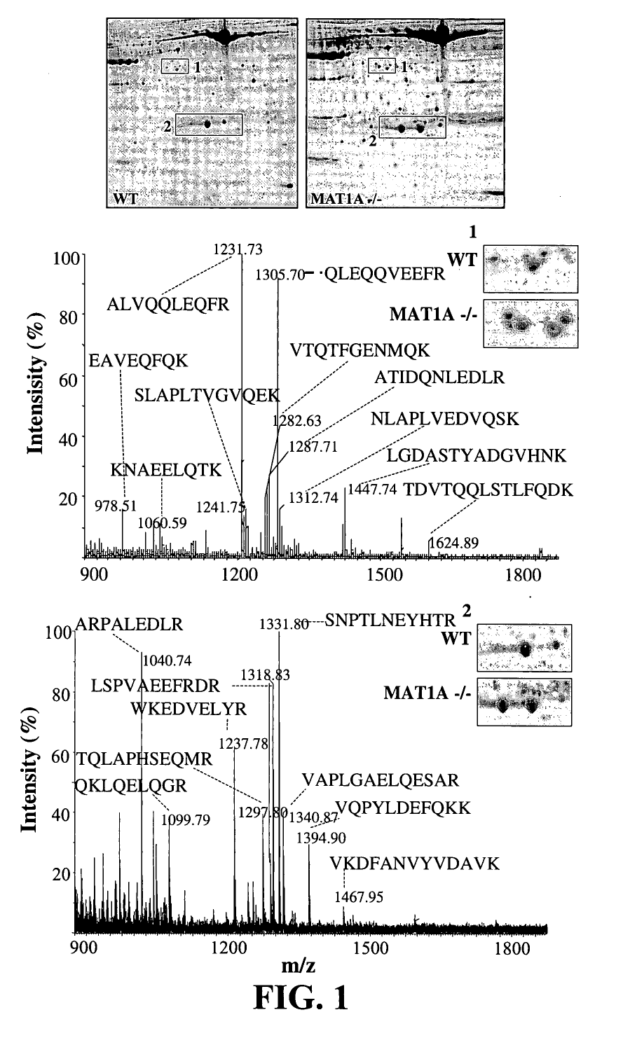

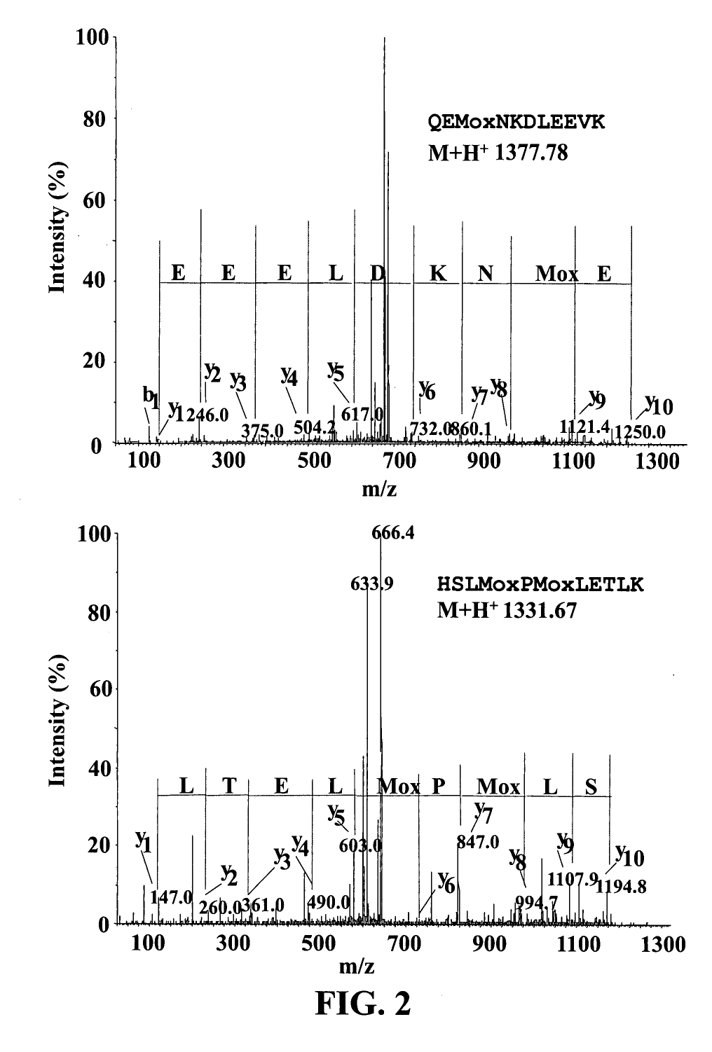

Analysis by means of PMF made it possible to identify these two increased proteic bands as apolipoprotein AIV and apolipoprotein AI (FIG. 1). Since Apo AI has been previously associated with liver diseases, this analysis was based on the molecular characterisation of the specifically increased form. Thus, through the tryptic digestion of the different apo AI isoforms, followed by nano-liquid chromatography coupled to a Q TOF micro-mass spectrometer by means of an electrospray ionisation source (ESI/MS/MS). The sequenced peptides represent over 40% of the Apo AI sequence in the three isoforms (Table 1). Four methionine residues oxidised to methionine sulfoxide were identified. Oxidation of M169 and M218 was common to the three Apo AI isotopes and, therefore, artifactual oxidation generated during sample analysis cannot be excluded. However, M85 and M216 sulfoxides were only identified in isoform 1 (FIG. 2). This specificity, confirmed in three independent analyses, suggests the physiological relevance of the selective oxidation of residues M85 and M216.

Table 1. Analysis of Apo AI isoforms in murine serum by means of LC/MS/MS. Tryptic peptides from Apo A-I isoforms from MATIA−/− serum were resolved by means of reverse-phase nanoflow liquid chromatography connected to an ESI/MS/MS mass spectrometer. The sequences of the Apo A-I peptides were deduced by means of de novo synthesis analysis. The oxidised methionine residues are highlighted.

| ApoA1 | | | |
|---|---|---|---|
| iso- | Sequenced | Oxidised | % Se- |
| forms | peptides | peptides | quenced |
| Apo | VKDFAVNYVDAVK | QEMNKDLEEVK | 44 |
| A-I_1 | DFWDNLEK | TQLAPHSEQMR | |
| | VQPYLDEFQK | HSLMPMLETLK | |
| | VAPLGAELQESAR | | |
| | SNPTLNEYHTR | | |
| | ARPALEDLR | | |
| Apo | VKDFAVNYVDAVK | TQLAPHSEQMR | 40 |
| A-I_2 | DFWDNLEK | HASLMPMLETLK | |
| | VQPYLDEFQK | | |
| | VAPLGAELQESAR | | |
| | QKLQELQGR | | |
| | SNPTLNEYHTR | | |
| | ARPALEDLR | | |
| Apo | VKDFAVNYVDAVK | TQLAPHSEQMR | 44 |
| A-I_3 | DFWDNLEK | HASLMPMLETLK | |
| | QEMNKDLEEVK | | |
| | VQPYLDEFQK | | |
| | VAPLGAELQESAR | | |
| | QKLQELQGR | | |
| | SNPTLNEYHTR | | |
| | ARPALEDLR | | |

### EXAMPLE 2

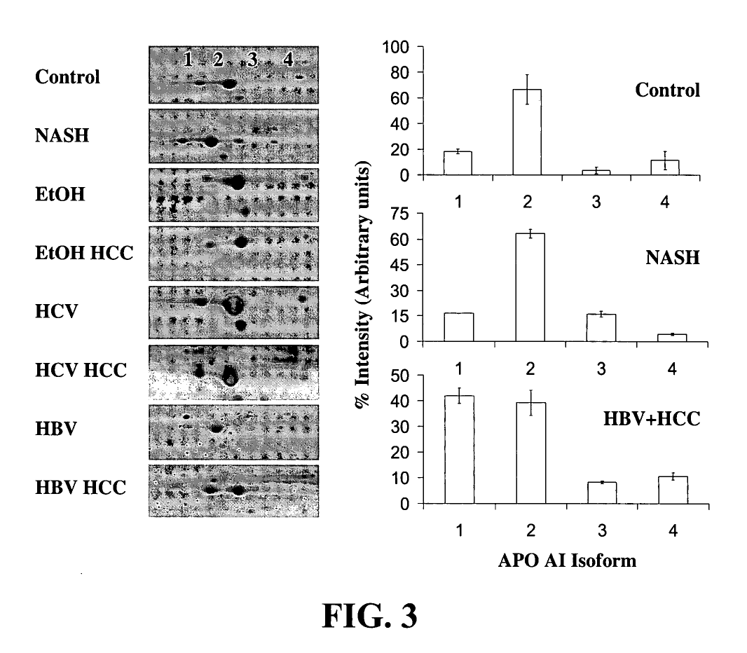

The levels and the post-translational state of Apo AI were studied in serum samples obtained from patients with different liver pathologies, including non-alcoholic steatohepatitis, alcoholic cirrhosis, HBV, HCV, and HCC. Four bands were identified as Apo AI by means of PMF (Peptide Mass Fingerprint) and de novo peptide sequencing in all the samples and 2D Western blot analysis. Through this study, pathology-specific alterations in the relative amount of Apo AI isoforms were detected. All the serum samples from patients with HBV and HCC showed a 2.5-fold increase in isoform 1, with respect to the controls (relative amount 41.93±3.00% as compared to 18.23±1.08% in control individuals) (FIG. 3).

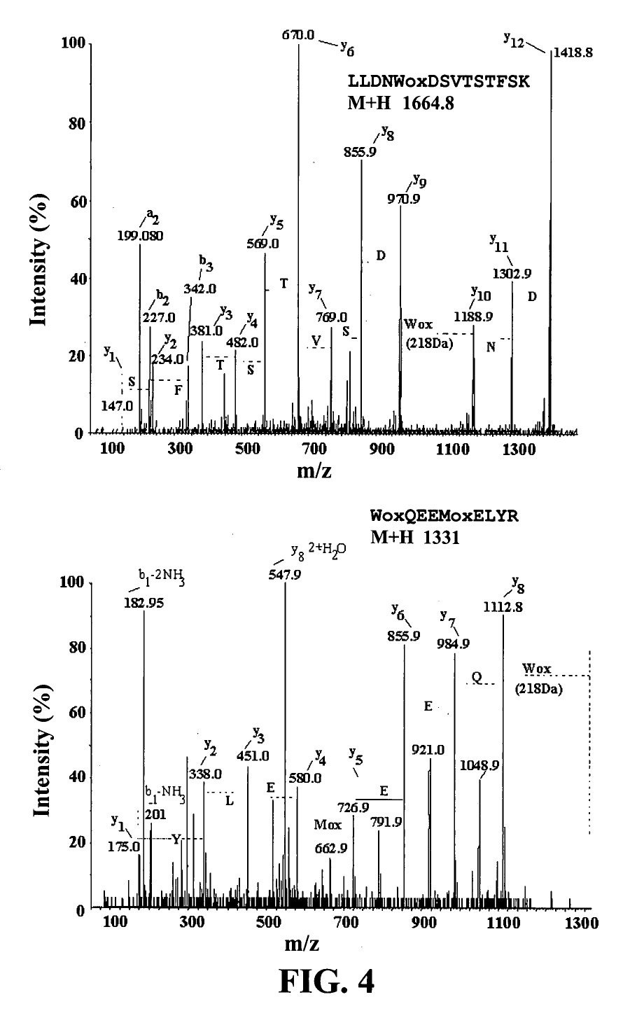

Subsequently, the decision was made to analyse whether the increase in the acidic isoform of Apo AI was the result of an increase in the protein's oxidation state. The products of the tryptic digestion of Apo AI isoforms from three different patients suffering from HBV+HCC were analysed by means of nano-liquid chromatography (nano LC) coupled to a Q TOF Micro-mass spectrometer by means of an electrospray ionisation source (ESI/MS/MS). Three specific oxidations were selectively detected in isoform 1. Oxidation of methionine 112 to methionine sulfoxide was evidenced by the +16 Da deviation of the ion's molecular mass (FIG. 4). Moreover, ions from tryptophan 50 and 108 residues showed addition of 32 mass units, which is consistent with the formation of formylknynurenine through two consecutive oxidation steps. M112 sulfoxide and formylknynurenine at positions 50 and 108 were only identified in isoform 1 (Table 2), while the sequence of the analogous peptides on isoforms 2, 3, and 4 showed the non-oxidised methionine and tryptophan residues. These data were consistent in three independent analyses, which supports the physiological relevance of these modifications.

Table 2. Analysis of Apo AI isoforms in human serum by means of LC/MS/MS. Tryptic peptides of Apo AI isoforms from human serum were resolved by means of a C18 reverse-phase nanoflow column connected to a Q TOF Micro-mass spectrometer (ESI/MS/MS). Apo AI peptide sequences were deduced from their fragmentation spectra. The oxidised methionine and tryptophan residues are highlighted.

| ApoA1 | | | |
|---|---|---|---|
| iso- | Sequenced | Oxidised | % Se- |
| forms | peptides | peptides | quenced |
| Apo | VKDLATVYVDVLK | LLDNWDSVTSTFSK | 51 |
| AI 1 | DLATVYVDVLK | WQEEMELYR | |
| | DYVSQFEGSALGK | | |
| | VQPYLDDFQK | | |
| | THLAPYSDELR | | |
| | LEALKENGGAR | | |
| | ATEHLSTLSEK | | |
| | AKPALEDLAR | | |
| | QGLLPVLESFK | | |
| | VSFLSALEEYTK | | |
| Apo | VKDLATVYVDVLK | | 45 |
| AI_2 | DYVSQFEGSALGK | | |
| | LLDNWDSVTSTFSK | | |
| | VQPYLDDFQK | | |
| | WQEEMELYR | | |
| | LHELQEK | | |
| | LEALKENGGAR | | |
| | ATEHLSTLSEK | | |
| | AKPALEDLAR | | |
| | QGLLPVLESFK | | |
| | VSFLSALEEYTK | | |
| Apo | DYVSQFEGSALGK | | 28 |
| AI_3 | LLDNWDSVTSTFSK | | |
| | VQPYLDDFQK | | |
| | WQEEMELYR | | |
| | THLAPYSDELR | | |
| | QGLLPVLESFK | | |
| Apo | DYVSQFEGSALGK | | 24 |
| AI_4 | LLDNWDSVTSTFSK | | |
| | VQPYLDDFQK | | |
| | THLAPYSDELR | | |
| | QGLLPVLESFK | | |

1-23. (canceled)

24. Method for diagnosing hepatic diseases wherein said method comprises detecting an increase in oxidised apolipoprotein AI isoforms in at least one tryptophan or methionine residue, or fragments thereof which contain at least one of said oxidised residues.

25. Method according to claim 24, wherein said method comprises the detection of the increase in at least one oxidised apolipoprotein AI isoform in at least one residue selected from W50, W108 and M112, or fragments thereof which contain at least one of said oxidised residues.

26. Method according to claim 25, wherein said method comprises the detection of the increase in at least one oxidised apolipoprotein AI isoform in any combination whatsoever of residues W50, W108 and M112, either two of them or all three.

27. Method according to claim 24, wherein said W50 and W108 residues are oxidised to kynurenine, formylkynurenine, hydroxytryptophan, or 3-OH-kynurenine.

28. Method according to claim 24, wherein said M112 is oxidised to M112 sulfoxide, or methionine sulfone.

29. Method according to claim 24, wherein said detection is performed on an isolated biological sample.

30. Method according to claim 24, wherein said biological sample is selected from: serum, plasma, blood, urine, saliva, cerebrospinal fluid, tears, amniotic fluid, tissue wash, tissue homogenate, and cell lysate.

31. Method according to claim 24, wherein the biological sample is serum.

32. Method according to claim 24, wherein said method comprises the detection in biological samples obtained from individuals suffering from hepatitis.

33. Method according to claim 32, wherein said method comprises the detection in biological samples obtained from individuals suffering from HBV.

34. Method according to claim 24, wherein said detection is performed by comparing with purified Apo AI standards in known quantities, or by comparing with the quantities of said isoform present in the same type of isolated biological samples from healthy individuals.

35. Method according to claim 24, wherein the detected increase in the oxidised isoform of Apo AI is at least 1.5-fold with respect to the control level.

36. Method according to claim 24, wherein the observed increase in the oxidised isoform with respect to the control level is at least 2.5-fold.

37. Method according to claim 24, wherein the presence of oxidised apo AI isoforms is detected and quantified by means of a method selected from: mass spectrometry, immunoassays, chemical assays, liquid chromatography, direct and indirect photometric methods and combinations thereof.

38. Method according to claim 37, wherein the mass spectrometry methods are selected from: tandem mass spectrometry coupled with liquid chromatography (LC-MS) and MALDI-TOF-MS (matrix-assisted laser desorption/ionization mass spectrometry time-of-flight).

39. Method according to claim 37, wherein said immunoassays are selected from: homogeneous assays, heterogeneous assays, enzyme immunoassays (EIA, ELISA), competition assays, immunometric assays (sandwich), turbidimetric assays, nephelometric assays and combinations thereof.

40. Method according to claim 37, wherein said assays comprise the use of antibodies, aptamers and/or lecithins, which specifically recognise said apo AI isoforms or their fragments.

41. Method according to claim 24, wherein said increase in apolipoprotein AI isoforms is related to the presence of hepatocellular carcinoma in the individual from whom the biological sample has been obtained.

42. Method according to claim 41, wherein said hepatocellular carcinoma is at an early stage.

43. Kit to determine the increase in the apolipoprotein AI isoform according to the procedure defined in claim 24, wherein said kit comprises the means to detect and quantify the presence of said isoform, or fragments thereof, in biological samples.

44. Kit to determine the increase in the apolipoprotein AI isoform according to claim 43, wherein said kit comprises reagents to perform the procedure disclosed in the present invention.

45. Kit to determine the increase in the apolipoprotein AI isoform according to claim 43, wherein said reagents are selected from: specific ligands of Apo AI isoforms, marker components to detect Apo AI isoforms, buffers, diluents, standards and controls.

46. Kit to determine the increase in the apolipoprotein AI isoform according to claim 43, wherein said kit additionally comprises bottles, phials, tubes, needles, solid substrates and instructions.
